(19) United States
(12) Patent Application Publication
(10) Pub. No.: US 2008/0262292 A1
(43) Pub. Date: Oct. 23, 2008

(54) Method and Device for Wireless Energy Transmission From a Magnet Coil System to a Working Capsule

(76) Inventors: Klaus Abraham-Fuchs, Erlangen (DE); Rainer Kuth, Hochstadt (DE); Johannes Reinschke, Nurnberg (DE); Rudolf Rockelein, Erlangen (DE); Sebastian Schmidt, Weisendorf (DE)

Correspondence Address: SCHIFF HARDIN, LLP;PATENT DEPARTMENT, 6600 SEARS TOWER, CHICAGO, IL, 60606-6473, US

(21) Appl. No.: 12/089,780

(22) PCT Filed: Sep. 29, 2006
(86) PCT No.: PCT/EP06/66928

§ 371 Date: Apr. 10, 2008

(30) Foreign Application Priority Data
Nov. 10, 2005 (DE) 10 2005 053 759.6

(51) Int. Cl. A61B 1/00

(52) U.S. Cl. 600/101

(57) ABSTRACT

In a method and device for wireless energy transmission between a magnetic coil system, having multiple excitation coils located outside of a patient, to a working capsule located in the patient, the working capsule has at least one induction coil, and a positioning device determines the position and orientation of the working capsule relative to the magnetic coil system. Using the position and orientation information, the magnetic coil system generates a first magnetic field that exerts a force on the working capsule at a location of the working capsule in the patient. The magnetic system also uses at least one of the position or the orientation to generate a second magnetic field for energy transmission to the working capsule at the location within the patient.

### BACKGROUND OF THE INVENTION

1. Field of the Invention

The invention concerns a method and a device for wireless energy transmission from a magnetic coil system outside of a patient to a working capsule having at least one induction coil in the patient. If multiple induction coils are present, these are aligned parallel to one another.

2. Description of the Prior Art

In medicine it is frequently necessary to execute a medical procedure (which, for example, can be a diagnosis or a treatment) inside a (normally living) person or animal as a patient. The target area of such a medical procedure is often a hollow organ in the appertaining patient, in particular his gastrointestinal tract. For a long time the medical procedures have been conducted with the aid of catheter endoscopes which are inserted into the patient from the outside in a non-invasive or minimally-invasive manner. Conventional catheter endoscopes hereby exhibit various disadvantages; for example, they cause pain in the patient or can reach remote internal organs only with difficulty or not at all.

Therefore, video capsules from the company Given Imaging which the patient swallows are known for catheter-free or wireless endoscopy, for example. The video capsule moves through the digestive tract of the patient due to peristalsis and hereby acquires a series of video images. These are transmitted to the outside and stored in a recorder. The patient can freely move during the multiple hours in which the capsule resides in the body since the patient takes corresponding reception antennas and a recorder with him or her on his or her body. The alignment of the capsule and therewith the viewing direction of the video images as well as the duration of residence in the body of the patient are random. The capsule has no active functionality except for the image acquisition. Diagnostic functions such as targeted observation, cleaning, biopsy are not possible, more are targeted treatments inside the patient, for example medicine administration. This is unacceptable or unsatisfactory for a complete diagnosis.

Lately it has become known (for example from DE 103 40 925 B3) to move magnetic bodies through hollow organs of a patient by means of magnetic, contact-free force transfer with the use of a magnetic coil system. The force application ensues in a targeted manner, without contact and controlled from the outside.

A magnetic body is, for example, a working capsule (also called an endocapsule or an endorobot) containing a permanent magnet. The working capsules exhibit functionalities of a conventional endoscope, for example video acquisition, biopsy or clips. A medical procedure can thus be implemented autarchically (i.e. wirelessly or without a catheter) with such a working capsule; no cable or mechanical connection from the working capsule to the exterior of the patient exists.

FIG. 3 shows a magnetic coil system 100 (known from DE 103 40 925 B3) that is described briefly in the following. DE 103 40 925 B3 is referenced for a more comprehensive, detailed description of the magnetic coil system 100 and its mode of operation. The magnetic coil system 100 has fourteen excitation coils 102a-n, of which only the excitation coils 102a-c, 102d and 102g-n are visible in FIG. 3. The six excitation coils 102a-f are thereby executed as rectangles and form the edges of a cuboid.

The remaining eight excitation coils 102g-n together form the generated surface of a cylinder embedded in the cuboid just described. Every single one of the excitation coils 102a-n is connected to a power supply 106 via a supply line 104a-n. For clarity only the supply lines 104a-c and 104e are shown in FIG. 3. A specific current strength with specific time curve (naturally in the scope of the capacity of the power supply 106) is impressed on each of the excitation coils 102a-n independent of one another via the power supply 106. Each of the excitation coils 102a-n thus generates its own magnetic field. A nearly arbitrary field distribution in terms of strength and direction can therewith be generated in the inner chamber 108 the magnetic coil system 100. A patient (not shown) is located in this inner chamber 108, and inside the body of this patient a working capsule 110 is located that contains a magnetic element (not shown), for example a permanent magnet.

A positioning device 112 is associated with the magnetic coil system 1001 which positioning device 112 detecting the attitude and orientation of the working capsule 110 in a coordinate system 114 associated with the magnetic coil system 100. The attitude of the working capsule 110 or the attitude of the geometric center of this is indicated by the dashed line 116 in FIG. 3. The orientation of the working capsule 110 is represented by the arrow 118 in FIG. 3 and is detected by the positioning device 112 relative to the coordinate system 114. The working capsule can exhibit an arbitrary (for example oblong or rotationally symmetrical) geometric shape. The orientation would then correspond to the direction of the unit vector in the longitudinal direction of the working capsule 1101 for example, The entire attitude of the working capsule 110 (thus in particular the center of gravity coordinates and the longitudinal axis direction) is thus completely described and known in the coordinate system 114.

The positioning device 112 transmits attitude and orientation information of the working capsule 110 to the power supply 106. The power supply 106 thereupon feeds the excitation coils 102a-n with current such that a magnetic field 120 (represented by the field lines 120 in FIG. 3) appears at the location of the working capsule 110. The magnetic field is designed so that it interacts with the permanent magnets in the working capsule 110 such that a desired force 122 and/or a desired torque (not shown) acts upon the working capsule 110. The working capsule 110 in the patient is moved, aligned and/or rotated in this manner.

All of the entire energy that the working capsule itself requires during the implementation of the medical procedure is provided by batteries or capacitors inside the working capsule, for example. The energy quantity is in particular limited by the limited size of such a working capsule (of, for example, 20 mm length and 10 mm diameter) and the other internal components. The functional duration or functional capability of the working capsule is likewise limited by the available energy quantity. Particularly power-intensive medical procedures such as, for example, hollow organ illumination, taking biopsies, thermal coagulation or laser applications can be implemented only in a limited manner or not at all.

In order to increase the available total energy for a working capsule it is known to wirelessly transmit energy from outside the patient to the working capsule inside the patient. A coil arrangement that can be worn like a jacket is known for this purpose from United States Patent Application Publication No. 2005/0065407 A1, which coil arrangement the patient wears on the body and from which energy is transmitted to the capsule inside the patient. The direction of the transmission field is constant; the reception coil must thus be correspondingly designed. A separate cooling of the transmission coils that is designed for this purpose is additionally provided in the wearable coil arrangement.

To reduce the transmission losses of the energy from the outside of the patient to the working capsule inside the patient, WO 02/080753 A2 suggests to locate the capsule inside the patient and to shift an external energy source (which surrounds the patient approximately in the shape of circular sectors) in the longitudinal direction of the patient to the level of the position of the capsule. Since the orientation of the capsule is unknown, orthogonal coils for receiving the energy are provided in the capsule in order to be able to always receive as much energy as possible in the capsule in every capsule orientation.

To improve the energy reception in the working capsule in a given external field, in DE 10 2004 034 444 A1 it is proposed to use for energy reception a number of reception elements that exhibit different directional dependencies with regard to the radiated fields. For example, ten differently oriented receiver coils are arranged inside the capsule in order to always ensure an optimal energy coupling in the capsule.

The more receiver coils that are to be provided in the working capsule, the smaller these must be individually designed if the total size of the capsule is to remain unchanged, However, since the energy feed into a coil depends on the coil surface, this consequently entails a reduction of the maximum energy or power that can be transmitted into coil (and thus into the working capsule).

### SUMMARY OF THE INVENTION

An object of the present invention is to provide a method and a device for improved wireless energy transmission from a magnetic coil system to a working capsule.

With regard to the method, the object is achieved by a method for wireless energy transmission from a magnetic coil system comprising multiple (in particular fourteen) excitation coils outside of a patient to a working capsule in the patient which has at least one induction coil of the same alignment as the excitation coils, wherein

a positioning device determines the position and orientation of the working capsule relative to the magnetic coil system and

using the position and orientation, the magnetic coil system generates a first magnetic field for force exertion on the working capsule at the location of the working capsule, in which:

the magnetic coil system generates a second magnetic field for energy transmission to the working capsule at the location of the working capsule using the position and/or orientation.

The magnetic coil system in accordance with the invention has a number of excitation coils that are able to generate the first magnetic field necessary for force exertion on the capsule, the exertion of a torque is also to be understood as force exertion. The first magnetic field can thus also be designated as a navigation magnetic field. The first inhomogeneity non-homogenous magnetic field is a gradient magnetic field of complicated geometry, which gradient magnetic field can be scaled in direction and strength. The coil system is therefore easily able to also generate the (normally homogeneous) second magnetic field for energy transmission, and in fact in any orientation relative to the magnetic coil system. The second magnetic field can also be designated as an induction magnetic field. Six Helmholtz coils arranged as a cylinder or cuboid are sufficient for such field generation.

The position and orientation of the working capsule must be known for the navigation, thus force exertion on the working capsule. A corresponding positioning device is thus present which determines the position and orientation of the induction coil relative to the magnetic coil system. Naturally, the attitude of the induction coil in the capsule must be known. In this simplest case the induction coil is therefore rigidly installed in the capsule. The positioning device is independent of the inductive energy transmission.

In particular the momentary orientation of the induction coil is therefore also known since its attitude in the working capsule is known. The direction in which the magnetic field for inductive energy transmission is to be generated is thus known to the magnetic coil system or its controller. Namely, the second magnetic field can always be generated so that it optimally crosses the induction coil, for example precisely along the coil axis. The power received in the induction coil is thus maximal at a given field strength. The excitation coils are therefore controlled so that a second magnetic field is generated which is optimally aligned relative to the induction coil.

Since a corresponding positioning device is present anyway in the cited magnetic coil system for contact-less force execution on the working capsule and the excitation coils for generation of the first low-frequency magnetic field are likewise present anyway, the excitation coils need only to be suitably controlled (thus in an alternative manner, i.e. with alternative current patterns) in order to generate the second magnetic field and thus to enable an energy transmission to the working capsule.

With regard to the energy feed into the working capsule, the excitation coils are likewise dimensioned for the generation of the first magnetic fields such that powers of a magnitude which cannot be generated by systems such as the jacket-like system from US 2005/0065407 can be generated easily. Corresponding power transmission stages and cooling devices are likewise present. A sufficiently large power can therewith be transmitted to the working capsule, which also enables power-intensive or energy-intensive medical procedures.

Since only a single induction coil must be present in the working capsule, this can be designed as large as possible; for example, it can cover the maximum capsule projection surface. Due to the maximum possible surface, the energy feed into the induction coil is therefore likewise as large as is now possible for a given size of the working capsule.

The magnetic coil system can generate first magnetic field and second magnetic field in first and second frequency ranges differing from one another. The frequency ranges can then in particular be executed so that they do not overlap, such that navigation and energy transmission are associated with separate frequency ranges. A mutual interference is thus precluded. Namely, the second magnetic field is not capable of setting the capsule into motion since this possesses no significant gradient portion at the capsule location and thus exerts no force on the capsule, and the moment of inertia of the capsule in connection with the relatively high frequency range of (for example) over 1000 Hz (1 kHz) ensures that the second magnetic field (which is on average immaterial over time) leads to a negligible jitter movements of the capsule due to the impressed torque.

Magnetic fields in a first frequency range between 0 Hz and 50 Hz, for instance, are particularly advantageous for force exertion on the working capsule. A second, non-overlapping, higher frequency range from 500 Hz to 10 kHz can then be used for the magnetic fields for energy transmission without interfering with those for force exertion and those of the magnetic measurement system. The frequency range from 500 Hz to 10 kHz is particularly suitable for transmission of the energy through human body tissue to the capsule at the given distances of approximately 20 to 60 cm between magnetic coil system and working capsule.

Due to the difference of the frequency ranges for the first and second magnetic fields for force exertion and for energy transmission, these mutually barely influence one another. For example, the second magnetic field for energy transmission can be selected to be high-frequency and the first for navigation can be selected to be low-frequency.

First magnetic field and second magnetic field can therefore be superimposed, This leads to the situation that an energy transmission to the capsule occurs simultaneously during the navigation or force exertion and movement of the working capsule through the patient, An energy storage in the capsule can thereby be avoided, for example. The installation space of the components for energy supply in the capsule becomes smaller or can be used for other internals which, for example, serve for the implementation of the medical procedure.

Alternatively, the second magnetic field can be generated temporally multiplexed with the first magnetic field. First and second fields are thus generated in temporal rotation and not simultaneously. The respective maximum power of the magnetic coil system is available both for the movement or, respectively, force exertion on the working capsule and for the energy transfer thereto. An excitation coil system that is sufficient for movement of the working capsule thus does not have to be dimensioned larger for energy transmission, but rather can be utilized in a temporally multiplexed manner.

For example, during the energy transmission the capsule then rests without force exertion in the patient. Due to correspondingly short time intervals between two energy transmissions, the energy storage in the capsule can be dimensioned such that this must only bridge the intervening time of the force exertion, A capacitor with small capacity and thus a smaller structural size is then sufficient, for example. The working capsule can also be utilized so that this only implements an energy-intensive medical procedure in the rest state.

The position and orientation of the induction coil relative to the magnetic coil system can be determined in various ways. One possibility is determination by an x-ray system, The patient is x-rayed during the implementation of the medical procedure, such that the capsule can be recognized in terms of its position and orientation on the x-ray image. Due to the high x-ray contrast of the capsule, the dose of the x-ray exposure for the patient can be kept very low. Naturally, a corresponding registration (thus knowledge of the positions relative to one another) of the coordinate systems of magnetic coil system and x-ray system is hereby necessary; corresponding solutions are known from the literature.

Thus no additional positioning devices must be installed in the capsule. The entire internal space of the capsule is available for other internal components.

A second alternative is the use of an electromagnetic measurement system. Only minimal internals (i.e. those with small space requirements) are necessary for this purpose, for example an electromagnetic transmission or reception device. These can be executed correspondingly small, such that they require only a small space in the working capsule.

In particular three positioning coils aligned orthogonal to one another which are used to determine the orientation of the induction coil can be present in the working capsule. Since, for their functioning, the positioning coils must consume barely any energy from an external magnetic field in order to implement the position detection, these can be designed distinctly smaller than the induction coil and thus require barely any space in the capsule.

The electromagnetic position measurement system can operate in a third frequency range (that is again different from the first frequency range and second frequency range) in order to interfere with none of the other systems. The electromagnetic position measurement system can in particular be operated with a frequency of at least 10 kHz. Alternatively, position measurement system and second magnetic field can be operated in alternation for inductive energy coupling.

In order to be able to control the excitation coils particularly well for generation of the magnetic fields for force exertion and energy transmission, the excitation coils can exhibit a plurality of taps and be operated via various taps. Different coils thus do not need to be provided for generation of the various fields; rather, a coil can be operated in different operating modes. A corresponding mounting and a cooling for the excitation coils thus only needs to be provided once.

With regard to the device, the object of the invention is achieved via a device for wireless energy transmission from a magnetic coil system possessing multiple (in particular fourteen) excitation coils outside of a patient to a working capsule possessing at least one induction coil in the patient. The device has a positioning device for determination of a position and orientation of the working capsule relative to the magnetic coil system. The device furthermore has a control unit controlling the magnetic coil system. The control unit thereby controls the magnetic coil system or, respectively, adjusts the currents flowing into the excitation coils such that the magnetic coil system generates a first magnetic field at the location of the working capsule for force exertion on the working capsule. For this purpose, the control unit uses the position and orientation of the working capsule that are determined by the positioning device. Moreover, for energy transmission to the working capsule the control unit controls the magnetic coil system such that this generates a second magnetic field at the location of the working capsule. The control unit also uses the determined position and orientation of the working capsule for this.

The advantages resulting from the inventive device have already been explained in connection with the inventive method.

The device can have an x-ray positioning system for determination of position and orientation of the working capsule as explained above.

Alternatively, for this purpose the device can have an electromagnetic positioning system, the working capsule can have three positioning coils aligned orthogonally to one another.

As described, the excitation coils also possess various taps via which they can be selectively operated, for example for generation of the first magnetic field and second magnetic field.

### BRIEF DESCRIPTION OF THE DRAWINGS

FIG. 1 illustrates a magnetic coil system for magnetic navigation and energy transmission in accordance with the invention.

FIG. 2 illustrates coil currents in an excitation coil of FIG. 1 for navigation and energy transmission (a) separately, (b) modulated on one another and (c) in a temporally multiplexed manner.

FIG. 3 illustrates a magnetic coil system for movement of a magnetic body in a patient according to the prior art.

### DESCRIPTION OF THE PREFERRED EMBODIMENTS

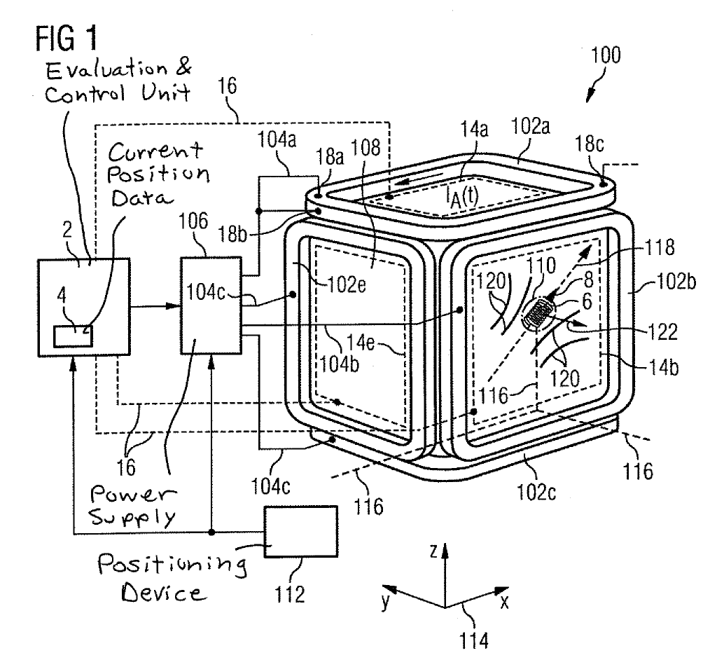

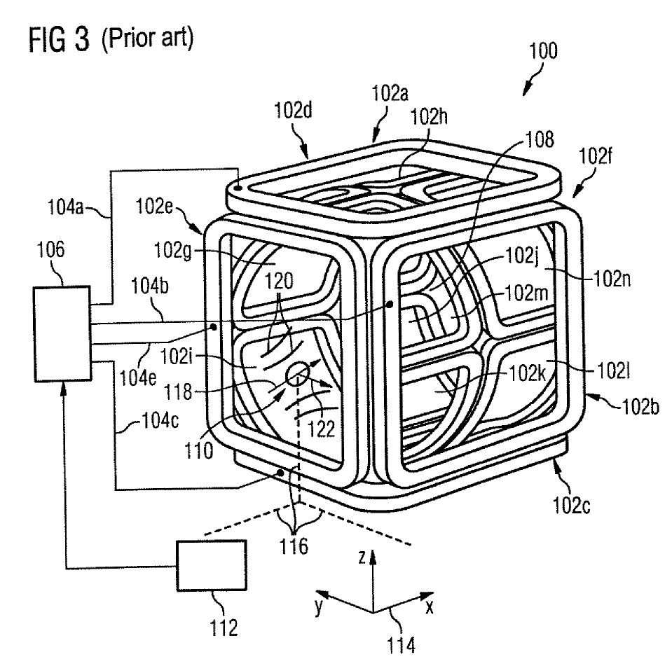

FIG. 1 shows the known magnetic coil system from FIG. 3 according to the prior art, expanded by an evaluation and control unit 2. The evaluation and control unit 2 receives from the positioning device 112 the current position data 4 of the working capsule 110 in the coordinate system 114 as well as target data for a new position and speed from an operator control device (not shown). The position data 4 are the attitude (lines 116) and orientation (arrow 118) of the working capsule 110 in the coordinate system 114, as explained in detail in connection with FIG. 3.

In contrast to FIG. 3, the working capsule 110 has an internal induction coil 6. For a given capsule geometry of the working capsule, this induction coil 6, together with the electrical consumer (not shown) connected to it, is designed such that it injects a greatest possible electrical power into the electrical consumer given a field distribution of the external magnetic field crossing it in the direction of its longitudinal axis. In the example from FIG. 1, the induction coil 6 is executed with the largest possible diameter, meaning that it abuts directly on the inner side of the outer casing of the working capsule 110. Since the attitude of the induction coil 6 in the working capsule 110 is fixed and known, the position data 4 likewise deliver position and orientation of the induction coil 6 to the evaluation and control unit 2.

The evaluation and control unit 2 calculates the currents I_{A}(t) through I_{N}(t) in the excitation coils 102a-n from the position data 4. Only I_{A}(t) is exemplarily plotted in FIG. 1. How the evaluation and control unit 2 controls the power supply 106 is indicated by the arrow 10, which power supply 106 then generates the actual currents I_{A}(t) through I_{N}(t) in the excitation coils 102a-n.

The currents I_{A}(t) through I_{N}(t) generate a magnetic field strength (indicated by the arrow 8) at the location of the induction coil 6 which induces the maximum possible electrical power in the induction coil 6. For example, this is provided for a field distribution in which the magnetic field strength is aligned parallel to the center longitudinal axis in the cylinder coil indicated in FIG. 1 as an induction coil 6.

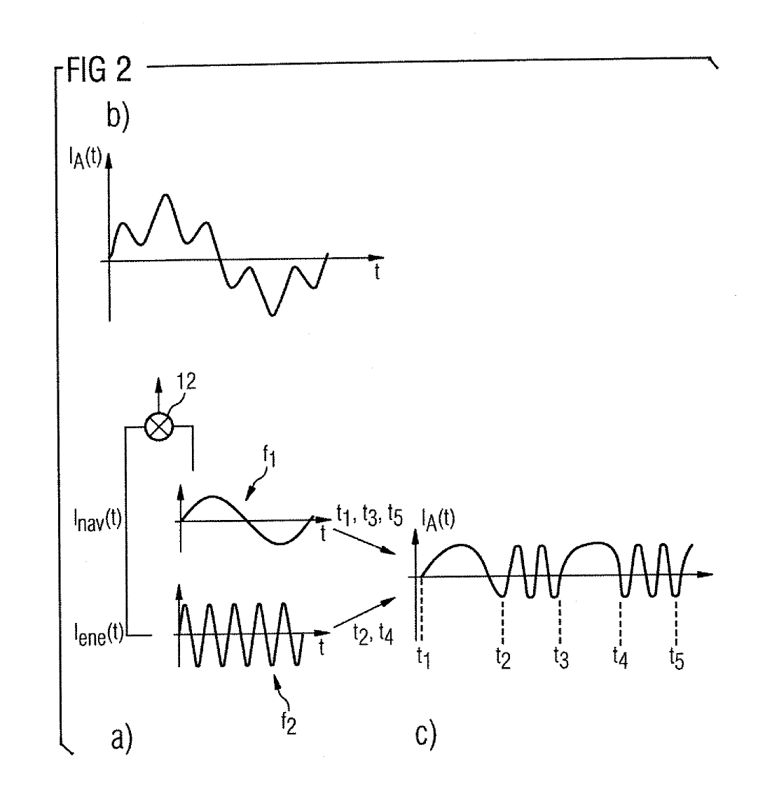

Illustration a) in FIG. 2 shows two temporal current curves Iₙₐᵥ(t) and Iₑₙₑ(t) whose sum is the current strength I_{A}(t) in the excitation coil 102a from FIG. 1. Iₙₐᵥ(t) is an exemplary temporal current strength curve for navigation of the working capsule 110 according to the prior art. The frequency f₁ of Iₙₐᵥ(t) lies in the range from 0-50 Hz. Iₑₙₑ(t) shows a temporal current curve for I_{A}(t) for generation of electrical energy in the induction coil 6. The working frequency f₂ of Iₑₙₑ(t) is 1-5 kHz.

Two alternatives for the actual feeding of current to the excitation coils 102a-n in the example of the excitation coil 102a are shown in illustrations b) and c) in FIG. 2. Illustration b) in FIG. 2 shows a current distribution I_{A}(t) in which the currents Iₙₐᵥ(t) and Iₑₙₑ(t) from FIG. 2 are superimposed, indicated by the adding unit 12.

The current feed or wiring of the excitation coils 102a-n hereby ensues via the taps 18a and 18b of each individual excitation coil 102a-n that are arranged at the ends of these, meaning that the entire excitation coil 102a-n has the current I_{A}(t) flowing through it. As described above, in FIG. 1 the taps 18a,b and c for the excitation coil 102a are shown only as examples.

The navigation (thus force exertion of the force 122 on the working capsule 110) as well as the energy feed of the capsule via injection of energy in the induction coil 6 ensue simultaneously with such a feed of current in FIG. 1 since both current patterns Iₙₐᵥ(t) and Iₑₙₑ(t) also flow simultaneously in the corresponding excitation coils 102a-n.

In contrast, illustration c) in FIG. 2 shows a time curve of the current I_{A}(t) in which the currents Iₙₐᵥ(t) and Iₑₙₑ(t) from illustration a) in FIG. 2 are switched in a temporal multiplex as a current I_{A}(t) to the excitation coil 102a.

The current Iₙₐᵥ(t) flows there from the point in time t1 until t2, the current Iₑₙₑ(t) flows between t2 and t3, Iₙₐᵥ(t) flows again between t3 and t4 etc. Navigation or, respectively, exertion of the force 122 on the working capsule 110 thus occur only in the time periods t1 through t2, t3 through t4 and after t5. By contrast, no force exertion on the working capsule 110 occurs in the time periods from t2 to t3 and t4 to t5; therefore injection of electrical energy into the induction coil 6 occurs, which just does not occur at the aforementioned time periods.

As described above, the feeding of current to the conductors of the excitation coils 102a-n now ensues only for the current Iₙₐᵥ(t) via the taps 18a and 19b of each individual excitation coil 102-n. The feeding of current Iₑₙₑ(t) ensues via the taps 18a and 18c. The tap 18c is hereby arranged centrally in the excitation coils 102a-n, for instance. Current Iₑₙₑ(t) thus flows through only a portion of the windings of the excitation coil 102a-n (each has approximately 100 to 200 windings). The excitation coils 102a-n then exhibit a suitable inductance or, respectively, resistance for this current pattern.

According to the previous description, the excitation coils 102a-n according to the prior art have been used both for direction of the navigation currents Iₙₐᵥ(t) and for the energy transmission currents Iₑₙₑ(t). As an alternative to this, in FIG. 1 the excitation coils 102a-n can also be used only in a more familiar manner for navigation (thus according to the prior art according to FIG. 3), thus are exclusively fed with navigation currents Iₙₐᵥ(t). Furthermore, these then serve solely for the exertion of force 122 on the working capsule 110.

For example, six cuboid or cylindrical induction transmission coils 14a-f (of which only 14a,b and 14e are visible in FIG. 1) are then additionally provided in the magnetic coil system 100. As an alternative to the manner described above, the induction transmission coils 14a-f are directly controlled by the evaluation and control unit 2 (thus not via the power supply 106), as indicated by the lines 16.

The induction transmission coils 14a-f serve exclusively for the inductive energy transmission to the working capsule 110 or, respectively, energy generation in the induction coil 6; currents Iₑₙₑ(t) thus flow through them.

The magnetic field direction (represented by the arrow 8) required for energy generation can in particular be realized by the six cuboid or cylindrically arranged excitation coils 102a-f or induction transmission coils 14a-f. Navigation and energy transmission to the capsule 110 do not mutually influence one another due to the different frequency ranges of the currents Iₙₐᵥ(t) and Iₑₙₑ(t).

Although modifications and changes may be suggested by those skilled in the art, it is the intention of the inventors to embody within the patent warranted heron all changes and modifications as reasonably and properly come within the scope of their contribution to the art.

We claim as our invention:

1-18. (canceled)

19. A method for wireless energy transmission from a magnetic coil system, comprising a plurality of excitation coils located outside of a patient, to a working capsule within the patient, said working capsule comprising at least one induction coil aligned with said excitation coils, said method comprising the steps of:
with an electronic positioning device, determining a position and an orientation of the working capsule relative to said magnetic coil system;
using said position and said orientation, automatically generating, with said magnetic coil system, a first magnetic field that exerts a force on said working capsule at a location of the working capsule within the patient; and
using at least one of said position and said orientation, generating, with said magnetic coil system, a second magnetic field for energy transmission to the working capsule at said location within the patient.

20. A method as claimed in claim 19 comprising generating, with said magnetic coil system, said first magnetic field in a first frequency range and generating said second magnetic field in a second frequency range differing from said first frequency range.

21. A method as claimed in claim 19 comprising, with said magnetic coil system, generating said first and second magnetic fields superimposed on one another.

22. A method as claimed in claim 19 comprising generating, with said magnetic coil system, said first and second magnetic fields temporally multiplexed relative to each other.

23. A method as claimed in claim 19 comprising determining said position and said orientation of said working capsule using an x-ray positioning system as said positioning device.

24. A method as claimed in claim 19 comprising determining said position and said orientation of said working capsule using an electromagnetic positioning system as said positioning device.

25. A method as claimed in claim 24 comprising, with said electromagnetic positioning system, determining said position and said orientation of said working capsule using three positioning coils that are orthogonal to each other in said working capsule.

26. A method as claimed in claim 24 comprising generating, with said magnetic coil system, said first magnetic field in a first frequency range and said second magnetic field in a second frequency range differing from said first frequency range, and comprising operating said electromagnetic positioning system in a third frequency range differing from said first and second frequency ranges.

27. A method as claimed in claim 19 wherein said excitation coils comprise a plurality of coil taps, and comprising, with said magnetic coil system, generating said first magnetic field via a first set of said taps and generating said second magnetic field using a second, different set of said taps.

28. A method as claimed in claim 19 comprising using only a portion of said excitation coils of said magnetic coil system for generating said second magnetic field.

29. A method as claimed in claim 19 wherein said magnetic coil system comprises additional induction transmission coils, and generating said second magnetic field using exclusively said additional induction transmission coils.

30. A device for wireless energy transmission from a magnetic coil system, comprising a plurality of excitation coils located outside of a patient, to a working capsule within the patient, said working capsule comprising at least one induction coil aligned with said excitation coils, said device comprising:
an electronic positioning device configured to determine a position and an orientation of the working capsule relative to said magnetic coil system;
a magnetic field gradient that, using said position and said orientation, automatically generates, with said magnetic coil system, a first magnetic field that exerts a force on said working capsule at a location of the working capsule within the patient; and
said magnetic field generator, using at least one of said position and said orientation, also generating, with said magnetic coil system, a second magnetic field for energy transmission to the working capsule at said location within the patient.

31. A device as claimed in claim 19 wherein said magnetic field generator is configured to generate, with said magnetic coil system, said first magnetic field in a first frequency range and to generate said second magnetic field in a second frequency range differing from said first frequency range.

32. A device as claimed in claim 19 wherein said magnetic field generator is configured to generate, with said magnetic coil system, said first and second magnetic fields superimposed on one another.

33. A device as claimed in claim 19 wherein said magnetic field generator is configured to generate, with said magnetic coil system, said first and second magnetic fields temporally multiplexed relative to each other.

34. A device as claimed in claim 19 wherein said positioning device is an x-ray positioning system.

35. A device as claimed in claim 19 wherein said positioning device is an electromagnetic positioning system.

36. A device as claimed in claim 24 wherein said electromagnetic positioning system comprises three positioning coils that are orthogonal to each other in said working capsule for determining said position and said orientation of said working capsule.

37. A device as claimed in claim 24 wherein said magnetic field generator is configured to generate, with said magnetic coil system, said first magnetic field in a first frequency range and said second magnetic field in a second frequency range differing from said first frequency range, and to operate said electromagnetic positioning system in a third frequency range differing from said first and second frequency ranges.

38. A device as claimed in claim 19 wherein said excitation coils comprise a plurality of coil taps, and wherein said magnetic field generator is configured to generate, with said magnetic coil system, said first magnetic field via a first set of said taps and generating said second magnetic field using a second, different set of said taps.

39. A device as claimed in claim 19 wherein said magnetic field generator is configured to use only a portion of said excitation coils of said magnetic coil system for generating said second magnetic field.

40. A device as claimed in claim 19 wherein said magnetic coil system comprises additional induction transmission coils, and generates said second magnetic field using exclusively said additional induction transmission coils.
